(19) United States
(12) Patent Application Publication
(10) Pub. No.: US 2009/0220578 A1
(43) Pub. Date: Sep. 3, 2009

(54) Hydrogel composition and method for making the same

(75) Inventor: Richard S. King, Warsaw, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: DePuy Products, Inc., Warsaw, IN (US)

(21) Appl. No.: 12/039,546

(22) Filed: Feb. 28, 2008

(51) Int. Cl. A61K 9/70; A61K 9/00

(52) U.S. Cl. 424/445; 424/486

(57) ABSTRACT

Disclosed is a hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN). The hydrogel of the present invention has an attractive combination of water absorption and compressive strength. The hydrogel composition finds use as a drug delivery device. The invention also provides a method of preparing such hydrogel composition.

### BACKGROUND OF THE INVENTION

The term “hydrogel” refers to a class of polymeric materials which are extensively swollen in an aqueous medium but which do not dissolve in them. Hydrogels have been proposed for a number of applications such as contact lenses, medical implant materials, stents, sutures, sustained or controlled release compositions, bandages, wound dressings, and cell growth matrices.

A number of polymeric materials have been considered for preparing hydrogel. In particular, polyethylene oxide has been proposed as a starting material for preparing hydrogels. For example, United States Patent Application Publication No. 2006/0074182-A1, filed Sep. 30, 2004, assigned to the same assignee as the present application, discloses, in an embodiment, a hydrogel composition comprising crosslinked polyethylene oxide and another hydrophilic polymer dispersed within the crosslinked polyethylene oxide network. The said another hydrophilic polymer is not crosslinked. The hydrogel composition is a semi-interpenetrating network (semi-IPN).

There exists a desire for hydrogel compositions comprising polyethylene oxide that combine attractive properties such as water content and compressive strength.

### BRIEF SUMMARY OF THE INVENTION

The present invention provides a hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN). The hydrogel of the present invention has an attractive combination of water absorption and compressive strength, which makes the hydrogel composition suitable for use in demanding applications such as human joints. The hydrogel composition is elastic and tough (or ductile). The invention also provides a method of preparing such hydrogel composition.

### DETAILED DESCRIPTION OF THE INVENTION

The present invention provides, in accordance with an embodiment, a hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN), i.e., a full IPN.

As indicated, the first crosslinked polymer comprises PEO. Any suitable PEO, e.g., one that is melt-processable, can be used as a precursor to the first crosslinked polymer. Preferably, the PEO is one that is melt-processable and radiation crosslinkable. As utilized herein, the term “melt-processable” refers to a polymer that can be processed in its molten state using processes such as injection molding, extrusion, blow molding, and/or compression molding. Preferably, a melt-processable polymer does not exhibit significant oxidative degradation, decomposition, or pyrolysis at the temperatures typically used in such molding processes. The term “radiation crosslinkable,” as utilized herein, refers to a polymer that forms crosslinks between individual polymer molecules and/or between segments of the same polymer molecule when the polymer is exposed to a suitable amount of radiation, such as gamma, x-ray, or electron beam radiation.

Any suitable PEO can be employed to create the crosslinked PEO. The PEO, in an embodiment, prior to crosslinking, has an average molecular weight of about 400,000 atomic mass units or more, for example, about 500,000 atomic mass units or more, about 1,000,000 atomic mass units or more (e.g., about 2,000,000 atomic mass units or more, about 3,000,000 atomic mass units or more, about 4,000,000 atomic mass units or more, or even about 7,000,000 atomic mass units or more). PEO's are different from polyethylene glycols in molecular weight. The term PEO refers to the high molecular weight material, i.e., one having a molecular weight of 50,000 or more.

The hydrogel composition of the invention comprises a second crosslinked polymer, which is preferably less hydrophilic than PEO. Any suitable hydrophilic polymer can be present as the second polymer, e.g., those selected from the group consisting of polyhydroxyethylmethacrylate (PHEMA), polyvinylpyrrolidone (PVP), polyvinyl alcohol, polyacrylic acid, polymethacrylic acid, polyhydroxyethylacrylate, poly(2-aminoethyl) methacrylate, and polyacrylamide, preferably the second crosslinked polymer comprises a hydrophilic polymer selected from the group consisting of polyhydroxyethylmethacrylate (PHEMA) and polyvinylpyrrolidone (PVP). In an embodiment, the second hydrophilic polymer is not kappa-carrageenan.

The hydrogel composition of the invention has a water content less than about 70% or more by weight, for example, about 20% to about 60% by weight, about 25% to about 55% by weight, about 30% to about 50% by weight of the composition at 20° C. and 1 atmosphere pressure. The water present in the hydrogel composition is preferably purified water such as RO water, deionized water, distilled water, and/or water treated with adsorbents such as activated carbon.

The invention also provides a drug delivery device comprising a hydrogel composition and an active ingredient dispersed or dissolved in the hydrogel composition. The drug delivery device can be any suitable device, for example, a medical implant, a wound dressing or a transdermal patch, cartilage repair and replacement, and nucleus pulposus replacement.

The invention further provides a method for producing a hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network IPN), said method comprising:

(a) providing a compression mold having an internal volume;

(b) filling at least a portion of the internal volume of the compression mold with polyethylene oxide;

(c) heat-compacting the polyethylene oxide within the compression mold to form a consolidated body therefrom;

(d) irradiating at least a portion of the consolidated body to crosslink at least a portion of the polyethylene oxide contained within the consolidated body;

(e) hydrating the consolidated body from (d);

(f) drying the hydrated consolidated body from (e) by lyophilization, by vacuum, or by heat/vacuum treatment to obtain a foam structure;

(g) imbibing the foam structure from (f) with a composition comprising water and/or polar solvent, a monomer corresponding to the second crosslinked polymer and a hydrophilic crosslinking agent to obtain a composite body;

(h) polymerizing the monomer and the hydrophilic crosslinking agent contained in the composite body; and

(i) hydrating the composite body from (h) to obtain the hydrogel composition.

As noted above, at least a portion of the internal volume of the compression mold (i.e., mold cavity) is filled with the PEO. After at least a portion of the internal volume of the compression mold is filled, the PEO contained within the compression mold is compressed for a time and under conditions (e.g., pressure and temperature) sufficient to form a consolidated body therefrom. It will be understood that the PEO is compressed by any suitable means, such as by mating the two halves of a two-part compression mold and applying an external force in a direction such that any substance contained within the mold (e.g., the PEO) is subjected to a compressive force.

Typically, the PEO is subjected to a pressure of about 3,400 kPa to about 28,000 kPa during the compression molding step. Preferably, the PEO is subjected to a pressure of about 3,800 kPa to about 14,000 kPa. During the compression molding step, the PEO typically is subjected to a temperature of about 70° C. to about 200° C. Preferably, the PEO is subjected to a temperature of about 90° C. to about 180° C., more preferably a temperature of about 120° C. to about 140° C., during the compression molding step. The PEO can be compressed in the compression mold for any amount of time sufficient to form a consolidated body therefrom. Typically, the PEO is compressed for about 5 to about 30 minutes, more preferably about 5 to about 10 minutes, during the compression molding step. It will also be understood that the particular time and conditions (e.g., pressure and temperature) necessary to form a consolidated body from the PEO will depend upon several factors, such as the type and/or amount of PEO, and the size (e.g., thickness) of the desired consolidated body, as well as molecular weight.

In accordance with an embodiment of the invention, the consolidated body is vacuum foil packaged and irradiated in (d) by exposing it to about 15 to about 100 KGy, preferably about 40 to about 60 KGy, of gamma radiation.

The consolidated body produced during the compression step can be irradiated using any suitable method, many of which are known in the art. For example, the consolidated body can be irradiated by exposing the mass to a suitable amount of gamma, x-ray, or electron beam radiation. Preferably, the consolidated body is irradiated by exposing it to about 15 to about 100 KGy of gamma radiation.

Preferably, the consolidated body is irradiated in an inert or reduced-pressure atmosphere. Irradiating the consolidated body in an inert (i.e., non-oxidizing) or reduced-pressure atmosphere reduces the effects of oxidation and chain scission reactions which can occur during irradiation in an oxidative atmosphere. Typically, the consolidated body is placed in an oxygen-impermeable package during the irradiation step. Suitable oxygen-impermeable packaging materials include, but are not limited to, aluminum, polyester coated metal foil (e.g., the Mylar® product available from DuPont Teijin Films), and multi-layer packages comprising polyethylene terephthalate and/or poly(ethylene vinyl alcohol). In order to further reduce the amount of oxidation which occurs during the irradiation of the consolidated body, the oxygen-impermeable packaging may be evacuated (e.g., the pressure within the packaging may be reduced below the ambient atmospheric pressure) and/or flushed with an inert gas (e.g., nitrogen, argon, helium, or mixtures thereof) after the consolidated body has been placed therein.

After the irradiation, the consolidated body is exposed to water to obtain a hydrated PEO gel. The consolidated body can be hydrated by any suitable technique. Typically, at least a portion of the consolidated body is submerged in an aqueous solution (e.g., de-ionized water, water filtered via reverse osmosis, a saline solution, or an aqueous solution containing a suitable active ingredient) for an amount of time sufficient to produce a hydrogel having the desired water content. For example, when a hydrogel comprising the maximum water content is desired, the consolidated body is submerged in the aqueous solution for an amount of time sufficient to allow the consolidated body to swell to its maximum size or volume. Typically, the consolidated body is submerged in the aqueous solution for at least about 50 hours, preferably at least about 100 hours, and more preferably about 120 hours to about 240 hours (e.g., about 120 hours to about 220 hours). The aqueous solution used to hydrate the consolidated body can be maintained at any suitable temperature. Typically, the aqueous solution is maintained at a temperature of at least about 25° C., more preferably at a temperature of about 30° C. to about 100° C., and most preferably at a temperature of about 40° C. to about 90° C. (e.g., about 50° C. to about 90° C., or about 50° C. to about 80° C.).

The hydrated PEO gel is dried by lyophilization, vacuum drying, or heat/vacuum drying to obtain a dry gel. The dry gel is immersed in an aqueous composition comprising water, optionally with a polar solvent, a monomer, and a crosslinking agent. The monomer can be present in any suitable concentration, for example, about 40% or more, such as from about 50% to about 80%, preferably from about 55% to about 75%, by weight of the composition. The crosslinking agent can be present in any suitable concentration, for example, about 1 ppm or more, such as from about 1 ppm to about 100 ppm, preferably from about 5 ppm to about 10 ppm by weight of the composition.

The monomer corresponding to the second crosslinked hydrophilic polymer is selected from the group consisting of 2-hydroxyethyl methacrylate, N-vinyl pyrrolidone, 2-aminoethyl methacrylate, and acrylamide, 2-hydroxybutyl methacrylate, 4-hydroxybutyl methacrylate, glycidyl methacrylate, glyceryl methacrylate, propylene glycol monomethacrylate, ethoxyethyl methacrylate, triethylene glycol methacrylate, 4- or 2-vinyl pyridine, and N, N-dimethyl acrylamide.

In accordance with an embodiment, the hydrophilic crosslinking agent is a bifunctional crosslinking agent, trifunctional crosslinking agent, or a tetrafunctional crosslinking agent. For example, the hydrophilic crosslinking agent is selected from the group consisting of N,N-methylene bisacrylamide, diethylene glycol dimethacrylate, triethylene glycol dimethacrylate, diethylene glycol acrylate, triethylene glycol acrylate, polyethylene glycol diacrylate, and polyethylene glycol dimethacrylate.

In accordance with an embodiment, the imbibed PEO gel is vacuum foil packaged and the monomer and the hydrophilic crosslinking agent are polymerized by exposing the composite body from (e) to about 15 to about 100 KGy, preferably about 40 to about 60 KGy, of gamma radiation. The radiation can be from a radioactive isotope such as ⁶⁰Co (gamma rays).

In accordance with another embodiment of the invention, the consolidated body in (g) further includes a free radical initiator. Any suitable free radical initiator can be included, for example, a peroxide initiator such as benzoyl peroxide or dicumyl peroxide, or an azo initiator such as AIBN. The monomer and the crosslinking agent can be polymerized by heating the composite body from (g).

Subsequent to the polymerization of the monomer and the crosslinking agent, the composite body in (h) is hydrated by submerging at least a portion of the composite body in an aqueous medium, which can include, in addition to water, additives such as therapeutic agents, humectants, salts, and/or surfactants. The invention also provides a hydrogel composition produced by the method described above.

The hydrogel composition of the invention can also contain any suitable additive, such as a therapeutic agent, surfactant, or humectant. The therapeutic agent can be a small molecule or a macromolecule. Examples of therapeutic agents include antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins, particularly bone growth factors and bone proteins.

Any suitable biocompatible surface active agent can be employed. Examples of surface active agents include phospholipids. Any suitable biocompatible humectants can be employed. Examples of suitable humectants include glycerin, propylene glycol, polyethylene glycol, glycol ethers such as diethylene glycol monomethyl ether and propylene glycol monomethyl ether.

The additive can be incorporated into the hydrogel composition by any suitable technique. For example, the additive can be incorporated (e.g., dry blended) into the PEO utilized to produce the hydrogel composition. Alternatively, the additive can be incorporated into the hydrogel composition during the hydration step, particularly the second hydration step. In particular, the hydrogel can be hydrated utilizing an aqueous and/or organic solution containing the additive. Alternatively, a hydrogel can be dehydrated or dried to produce a xerogel, and then the xerogel can be hydrated utilizing an aqueous and/or organic solution containing the additive.

The hydrogel composition of the invention can have a compressive strength greater than 1 MPa. In a preferred embodiment, the hydrogel composition of the invention has a compressive strength of about 1.5 MPa or more. In a more preferred embodiment, the hydrogel composition of the invention has a compressive strength of about 2 MPa or more, most preferably 2.1 to about 3.0 MPa. In accordance with an embodiment, the hydrogel composition has a water content of about 40% to about 50% and a compressive strength of 2 to about 2.6 MPa.

As utilized herein, the term “compressive strength” refers to the maximum resistance to fracture of a material (e.g., the hydrogel composition) under compressive stress. The compressive strength of the hydrogel composition can be measured using any suitable technique. One suitable technique for determining the compressive strength of a hydrogel is the method of C. Tranquilan-Aranilla et al., which is described in the article “Kappa-carrageenan-polyethylene oxide hydrogel blends prepared by gamma irradiation,” Radiation Physics and Chemistry 55:127-131 (1999). Preferably, the compressive strength of the hydrogel composition is determined using a modified version of the technique developed by C. Tranquilan-Aranilla et al. in which the sample used for the test is changed to measure approximately 15.9 mm (0.625 inches) in diameter and about 6.4 mm (0.25 inches) in thickness. The crosshead speed used in the modified technique is approximately 10 mm/min (0.4 inches/min), which is the same crosshead speed used by C. Tranquilan-Aranilla et al. The compressive strength of a hydrogel is considered to be within the ranges set forth herein when determined using the aforementioned preferred technique (i.e., the modified technique based upon the method of C. Tranquilan-Aranilla et al.).

The method of the invention can further comprise the step of sterilizing the hydrogel composition using any suitable process. The hydrogel composition can be sterilized at any suitable point, but preferably is sterilized after the composite body/hydrogel precursor is hydrated. Suitable non-irradiative sterilization techniques include, but are not limited to, gas plasma or ethylene oxide methods known in the art. For example, the hydrogel composition can be sterilized using a PlazLyte® Sterilization System (Abtox, Inc., Mundelein, Ill.) or in accordance with the gas plasma sterilization processes described in U.S. Pat. Nos. 5,413,760 and 5,603,895, which are incorporated by reference. In some circumstances, such as when the interior portions of the hydrogel composition require sterilization, the hydrogel composition can be sterilized using gamma irradiation at a relatively low dose of approximately 15 KGy to about 40 KGy using methods known in the art.

The hydrogel composition produced by the method of the invention can be packaged in any suitable packaging material. Desirably, the packaging material maintains the sterility of the hydrogel composition until the packaging material is breached.

The following examples further illustrate the invention but, of course, should not be construed as in any way limiting its scope.

### EXAMPLE 1

This example demonstrates the production of a hydrogel composition according to the invention.

A poly(ethylene oxide) having an average molecular weight of approximately 7 million atomic mass units (POLYOX™ WSR-303 poly(ethylene oxide) (available from The Dow Chemical Company, Midland, Mich.)) is placed into a compression mold having an internal volume. The internal volume of the compression mold defines a disk measuring approximately 89 mm (3.5 inches) in diameter and approximately 3.2 mm (0.125 inches) in thickness, and the PEO completely fills the compression mold. The PEO is packed into the compression mold by subjecting the consolidated body to a compressive force of approximately 2700 kPa (400 psi) for approximately 2-3 minutes at room temperature. Following the packing step, the temperature within the compression mold is increased from room temperature to approximately 127° C. (260° F.) at a rate of approximately 3.3° C./min (6° F./min) while the pressure on the compression mold is increased from 2700 kPa (400 psi) to about 11,000 kPa (1600 psi). The temperature and pressure within the compression mold are then maintained at approximately 127° C. (260° F.) and 11,000 kPa (1600 psi) for approximately 15 minutes. Following the heat-compacting step, the resulting consolidated disk is cooled from 127° C. (260° F.) to room temperature (i.e., approximately 22° C. (72° F.)) at rate of approximately 3.9° C./min (7° F./min) while the pressure is reduced from 11,000 kPa (1600 psi) to approximately 480 kPa (70 psi).

The consolidated disk is vacuum packed in a foil, and is subjected to gamma irradiation to a dose of 50 KGy. The irradiated disk is immersed in water for 72 hours or longer at room temperature. The hydrated disk is dried by lyophilization to obtain a dry PEO gel. The dry PEO gel is soaked in an aqueous solution containing a hydrophilic monomer and a crosslinking agent. The PEO gel soaked with the aqueous solution is packaged in vacuum foil and gamma irradiated to a dose of 50 KGy. The irradiated gel is soaked in water to obtain a hydrated gel, which is later imbibed with a monomer and crosslinking agent, followed by irradiation, as set forth below.

Table 1 below sets forth the properties of three PEO IPN's in accordance with an embodiment of the invention. The IPN's have attractive properties such as high compressive strength and high compressive modulus while maintaining water absorption.

TABLE 1
Composition and properties of PEO IPN's

| | | | Compressive | Strain | |
|---|---|---|---|---|---|
| | | Crosslinker, | strength, | to | Compressive |
| hydrogel | HEMA/water | ppm | MPa | failure, % | modulus, MPa |
| PEO | — | None | 0.5-0.6 | 61-80 | 0.08 |
| PEO-PHEMA | 70/30 | 10 MBA | 2.6 | 18 | 2.8 |
| IPN 1 | | | | | |
| PEO-PHEMA | 70/30 | 5 MBA | >2.0* | >75* | 0.2 |
| IPN 2 | | | | | |
| PEO-PHEMA | 70/30 | 5 DEGDMA | >1.9* | >78* | 0.2 |
| IPN 3 | | | | | |

MBA = methylene bis(acrylamide);
DEGDMA = diethyleneglycol dimethacrylate;
*symbol “>” denotes that the samples do not fail under the test conditions.

### EXAMPLE 2

This Example illustrates the water content of hydrogels prepared in accordance with another embodiment of the invention. The water content of PEO crosslinked by 50 KGy gamma radiation is 93.4%. The water content of semi-IPN polyethylene oxide—polyhydroxyethyl methacrylate is 51.7%. The semi-IPN hydrogel is fabricated using the same procedure as the two IPN hydrogels above except that there is no a crosslinker: 70/30 HEMA/water and 50 KGy gamma treatment. The water content of IPN polyethylene oxide—polyhydroxyethyl methacrylate (HEMA/water/N,N-methylene bisacrylamide —70/30/0.001) is 48.2%. Polymerization and crosslinking of PHEMA are by radiation treatment at 50 KGy. The water content of IPN polyethylene oxide—polydroxyethyl methacrylate (diethylene glycol dimethacrylate—70/30/0.001) is 43.2%, wherein polymerization and crosslinking of PHEMA are by radiation treatment at 50 KGy. The foregoing shows that a hydrogel in accordance with an embodiment of the invention has an attractive combination of properties—water content and compressive strength.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN).

2. The hydrogel composition of claim 1, wherein the second crosslinked polymer comprises a hydrophilic polymer selected from the group consisting of polyhydroxyethylmethacrylate (PHEMA), polyvinylpyrrolidone (PVP), polyvinyl alcohol, polyacrylic acid, polymethacrylic acid, polyhydroxyethylacrylate, poly(2-aminoethyl) methacrylate, and polyacrylamide.

3. The hydrogel composition of claim 2, wherein the second crosslinked polymer comprises a hydrophilic polymer selected from the group consisting of polyhydroxyethylmethacrylate (PHEMA) and polyvinylpyrrolidone (PVP).

4. The hydrogel composition of claim 1, which has a water content less than about 70% or more by weight at 20° C. and 1 atmosphere pressure.

5. The hydrogel composition of claim 1, which has a water content of about 20% to about 60% by weight at 20° C. and 1 atmosphere pressure.

6. A drug delivery device comprising a hydrogel composition and an active ingredient dispersed or dissolved in the hydrogel composition, wherein the hydrogel composition comprises water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN).

7. The drug delivery device of claim 6, which is a wound dressing.

8. A method for producing a hydrogel composition comprising water, a first crosslinked polymer, and a second crosslinked polymer, wherein the first crosslinked polymer comprises poly(ethylene oxide) and the second crosslinked polymer comprises a hydrophilic polymer other than poly(ethylene oxide), wherein the first crosslinked polymer and the second crosslinked polymer form an interpenetrating network (IPN), said method comprising:
(a) providing a compression mold having an internal volume;
(b) filling at least a portion of the internal volume of the compression mold with polyethylene oxide;
(c) heat-compacting the polyethylene oxide within the compression mold to form a consolidated body therefrom;
(d) irradiating at least a portion of the consolidated body to crosslink at least a portion of the polyethylene oxide contained within the consolidated body;
(e) hydrating the consolidated body from (d);
(f) drying the hydrated consolidated body from (e) by lyophilization, vacuum, or heat/vacuum to obtain a foam structure;
(g) imbibing the foam structure from (f) with a composition comprising water and/or polar solvent, a monomer corresponding to the second crosslinked polymer and a hydrophilic crosslinking agent to obtain a composite body;
(h) polymerizing the monomer and the hydrophilic crosslinking agent contained in the composite body; and
(i) hydrating the composite body from (h) to obtain the hydrogel composition.

9. The method of claim 8, wherein the polyethylene oxide has a molecular weight of about 1,000,000 or more.

10. The method of claim 8, wherein polyethylene oxide is compressed at a pressure of about 3,800 kPa to about 14,000 kPa.

11. The method of claim 8, wherein the polyethylene oxide is heated to a temperature of about 120° C. to about 140° C. during the compression.

12. The method of claim 8, wherein the consolidated body is irradiated in (d) by exposing it to about 15 to about 100 KGy of gamma radiation.

13. The method of claim 8, wherein the monomer in (g) is selected from the group consisting of hydroxyethyl methacrylate, N-vinyl pyrrolidone, 2-aminoethyl methacrylate, and acrylamide.

14. The method of claim 8, wherein the hydrophilic crosslinking agent is a bifunctional crosslinking agent, trifunctional crosslinking agent, or a tetrafunctional crosslinking agent.

15. The method of claim 8, wherein the hydrophilic crosslinking agent is selected from the group consisting of N,N-methylene bisacrylamide, diethylene glycol dimethacrylate, triethylene glycol dimethacrylate, diethylene glycol acrylate, triethylene glycol acrylate, polyethylene glycol diacrylate, polyethylene glycol dimethacrylate.

16. The method of claim 8, wherein the monomer and the hydrophilic crosslinking agent are polymerized by exposing the molded body from (e) to about 15 to about 100 KGy of gamma radiation.

17. The method of claim 8, wherein the consolidated body in (g) further includes a free radical initiator.

18. The method of claim 17, wherein monomer and the crosslinking agent are polymerized by heating the composite body from (g).

19. The method of claim 8, wherein the composite body in (h) is hydrated by submerging at least a portion of the composite body in an aqueous medium.

20. The method of claim 18, wherein the aqueous medium comprises, in addition to water, an active ingredient, a humectant, a salt, or a surfactant.

21. A hydrogel composition produced by the method of claim 8.
